SEQUENCE LISTING (23 sequences)

SEQ ID NO: 1 - RNA, 22 nt, Artificial Sequence
  Artificial sequence- cfa-miR-30b
uguaaacauccuacacucagcu

SEQ ID NO: 2 - RNA, 24 nt, Artificial Sequence
  Artificial sequence- cfa-miR-30d
uguaaacauccccgacuggaagcu

SEQ ID NO: 3 - RNA, 21 nt, Artificial Sequence
  Artificial sequence- cfa-miR-128
ucacagugaaccggucucuuu

SEQ ID NO: 4 - RNA, 22 nt, Artificial Sequence
  Artificial sequence- cfa-miR-133a
uugguccccuucaaccagcugu

SEQ ID NO: 5 - RNA, 22 nt, Artificial Sequence
  Artificial sequence- cfa-miR-133b
uuugguccccuucaaccagcua

SEQ ID NO: 6 - RNA, 21 nt, Artificial Sequence
  Artificial sequence- cfa-miR-142
cccauaaaguagaaagcacua

SEQ ID NO: 7 - RNA, 22 nt, Artificial Sequence
  Artificial sequence- cfa-miR-206
uggaauguaaggaagugugugg

SEQ ID NO: 8 - RNA, 22 nt, Artificial Sequence
  Artificial sequence- cfa-miR-320
aaaagcuggguugagagggcga

SEQ ID NO: 9 - RNA, 23 nt, Artificial Sequence
  Artificial sequence- cfa-miR-423a
ugaggggcagagagcgagacuuu

SEQ ID NO: 10 - RNA, 21 nt, Artificial Sequence
  Artificial sequence- cfa-miR-499
uuaagacuugcagugauguuu

SEQ ID NO: 11 - RNA, 22 nt, Artificial Sequence
  Artificial sequence- cfa-let-7b
ugagguaguagguugugugguu

SEQ ID NO: 12 - RNA, 22 nt, Artificial Sequence
  Artificial sequence- cfa-let-7e
ugagguaggagguuguauaguu

SEQ ID NO: 13 - RNA, 22 nt, Artificial Sequence
  Artificial sequence- hsa-let-7i-5p
ugagguaguaguuugugcuguu

SEQ ID NO: 14 - RNA, 22 nt, Artificial Sequence
  Artificial sequence- hsa-miR-29a-3p
uagcaccaucugaaaucgguua

SEQ ID NO: 15 - RNA, 22 nt, Artificial Sequence
  Artificial sequence- hsa-miR-486-5p
uccuguacugagcugccccgag

SEQ ID NO: 16 - RNA, 23 nt, Artificial Sequence
  Artificial sequence- hsa-miR-17-5p
caaagugcuuacagugcagguag

SEQ ID NO: 17 - RNA, 22 nt, Artificial Sequence
  Artificial sequence- cfa-miR-130b
cagugcaaugaugaaagggcau

SEQ ID NO: 18 - RNA, 23 nt, Artificial Sequence
  Artificial sequence- cfa-miR-20a
uaaagugcuuauagugcagguag

SEQ ID NO: 19 - RNA, 19 nt, Artificial Sequence
  Artificial sequence- cfa-miR-23a
aucacauugccagggauuu

SEQ ID NO: 20 - RNA, 22 nt, Artificial Sequence
  Artificial sequence- cfa-miR-26a
uucaaguaauccaggauaggcu

SEQ ID NO: 21 - RNA, 22 nt, Artificial Sequence
  Artificial sequence- oan-miR-7417-5p
uuccccacucugagcacacagc

SEQ ID NO: 22 - RNA, 23 nt, Artificial Sequence
  Artificial sequence- cel-mir-70-3p
uaauacgucguugguguuuccau

SEQ ID NO: 23 - RNA, 22 nt, Artificial Sequence
  Artificial sequence- ath-mir167d
ugaagcugccagcaugaucugg

(19) United States
(12) Patent Application Publication
(10) Pub. No.: US 2023/0332235 A1
(43) Pub. Date: Oct. 19, 2023

(54) BIOMARKERS FOR DIAGNOSING A DISEASE SUCH AS HEART OR CARDIOVASCULAR DISEASE

(71) Applicant: SRUC, Edinburgh (GB)

(72) Inventor: Eve HANKS, Edinburgh (GB)

(73) Assignee: SRUC, Edinburgh (GB)

(21) Appl. No.: 18/044,283

(22) PCT Filed: Sep. 9, 2021
(86) PCT No.: PCT/GB2021/052339

§ 371 Date: Mar. 7, 2023

(30) Foreign Application Priority Data
Sep. 9, 2020 (GB) 2014190.9

(51) Int. Cl. C12Q 1/6883; C12Q 1/6827; G16B 20/00; G16B 40/20

(52) U.S. Cl. CPC C12Q 1/6883; C12Q 1/6827; G16B 20/00; G16B 40/20; C12Q 2600/178; C12Q 2600/158

(57) ABSTRACT

A method is provided for detecting the presence of heart disease in a subject, comprising the steps of: (a) determining the level of expression of each of a plurality of miRNAs within a sample from a subject; and (b) using one or more Artificial Intelligence (AI) model to predict the disease condition of the subject.

The present invention relates to isolated nucleic acid molecules known as microRNAs (miRNAs) and miRNA precursor molecules and their use in diagnosis and therapy. The invention also relates to a method and a kit for diagnosing a disease such as heart or cardiovascular disease.

Biomarkers have the potential to allow for early diagnosis, risk stratification and therapeutic management of various diseases. Although research into the use of biomarkers has developed in recent years, the clinical translation of disease biomarkers as endpoints in disease management and in the development of diagnostic products still poses a challenge.

miRNAs are a class of small non-coding RNAs which have been identified as having the potential to act as biomarkers. miRNAs were first discovered in the free-living nematode Caenorhabditis elegans where it was found that small, non-coding RNAs known as lin-4 and let-7 were responsible for regulating the expression of developmental proteins in C. elegans through suppression of messenger RNA (mRNA) levels (Wightman, et al., 1993; Lee, et al., 1993; Lee & Ambros, 2001). miRNAs bind predominantly to the three prime (3′) untranslated region (UTR) of their target genes resulting in suppression of translation and/or mRNA degradation. Coutinho et al (2007) analysed bovine immunity and embryonic tissues and reported that miRNAs are frequently conserved across species. In addition, it was found that some miRNAs are expressed preferentially in specific tissue types while others are expressed more uniformly across different tissues.

miRNAs have been identified as key regulators of the immune system of many organisms (Mehta & Baltimore, 2016). They are recognised as key mediators of innate immunity (Momen-Heravi & Bala, 2018), the first line of defence, and adaptive immunity (Jia, et al., 2014) which is a specific response to a pathogen. This makes the use of miRNAs particularly interesting since understanding their expression will allow for a greater understanding of the epigenetic responses to disease, wherein the diseases are both infectious and non-infectious in origin (Rupaimoole & Slack, 2017). It was subsequently discovered that miRNAs are released from tissues into the systemic circulation and can be found in other biofluids (for example, in a blood sample). The term ‘liquid biopsy’ was thus adopted (Giannopoulou, et al., 2019). Furthermore, miRNAs also offer a potential as therapeutic targets. If miRNAs are dysregulated in disease states then it is considered that controlling their expression and encouraging healing over inflammation would be beneficial for patients. This idea has been termed anti-miRNAs (Piotto, et al., 2018).

Heart disease is common in dogs and cats with some breeds predisposed to certain conditions. There are a wide variety of heart diseases and each will benefit from a different treatment regime. Estimates on the proportion of cats and dogs affected by cardiovascular disease are 10-15% and 10%, respectively.

Current methods of detecting heart disease rely on assessing changes in the structure and/or function of the heart. Investigation to determine whether heart disease is present often involves an ECG, X-ray, ultrasound and/or a blood test to show if there has been any cardiac damage. A combination of these tests is often required for diagnosis which can be costly, invasive and stressful for the patient. In addition, the requirement for using these tests can often also represent a substantial delay in treatment.

miRNA profiles are thought to hold substantial amounts of information and are conserved across species such as farm animals, horses, companion animals and humans. So far, miRNAs have been mainly studied in tissue material where it has been found that miRNAs are expressed in a highly tissue-specific manner. In order to improve the biomarker capabilities in diagnosis there is a need for disease specific, well performing biomarkers such as miRNA biomarkers.

The present application aims to address the above problems.

According to a first aspect, there is provided a method for detecting the presence of heart disease in a subject, comprising the steps of:
(a) determining the level of expression of each of a plurality of miRNAs within a sample from a subject; and
(b) using one or more Artificial Intelligence (AI) model to predict the disease condition of the subject.

Preferably, the one or more AI model compares the level of expression of each miRNA molecule with at least one pre-determined reference level characteristic of a non-diseased subject for each one of the plurality of the miRNA molecules of step (a), wherein a deviation of the level of expression of said miRNA molecules from step (a) in comparison with the at least one reference level allows for the diagnosis and/or prognosis of the disease.

Preferably, the plurality of miRNA molecules comprise cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p and hsa-miR-486-5p.

Preferably, the subject is an animal. Typically, the subject is a cat or a dog.

It is an advantage of the invention that the method provides an accurate and useful test that can be used in veterinary practice. It is known that certain levels of expression of certain miRNA molecules can indicate the presence of heart disease. However, measuring the level of expression of the plurality of miRNA molecules in accordance with the invention allows for the accurate diagnosis of disease within a subject. The determination of disease within the context of the present invention would not be possible with one biomarker because it is not simply the increase or decrease of one marker that provides the diagnostic information. Rather, it is the differential expression of the plurality of miRNAs in relation to each other and the pattern recognition of the plurality of miRNAs that enables the disease detection.

It is another advantage of the invention that the method provides a test that can be carried out over a 15 to 30 minute time scale.

Preferably, the method further comprises the step of using a machine learning algorithm for predictive modelling. Advantageously, the use of predictive modelling allows for prediction of the presence or absence of disease within a subject.

Preferably, the method comprises the use of a combination of AI models. It is an advantage of the present invention that the use of a combination of AI models allows for the accurate determination of the presence or absence of disease in a subject.

Typically, the method further comprises the use of at least one normaliser and/or control miRNA molecule. Preferably, the control miRNA molecule is an off-species control miRNA molecule.

Preferably, the at least one normaliser is selected from the group consisting of hsa-miR-17-5p, cfa-miR-130b, cfa-miR-20a, cfa-miR-23a and/or cfa-miR-26a. Preferably, the at least one off-species control is selected from the group consisting of oan-miR-7417-5p, cel-mir-70-3p and/or ath-mir167d.

Preferably, at least one normaliser is used to ‘normalise’ data, i.e. to control for variation between the samples tested in the method of the invention, and the at least one control is used to try to ensure there are no failure or false readings in the results. Preferably, at least one off-species control is added in to show that the miRNAs detected are relevant to the dog and/or cat panel. Preferably, the off-species control is an miRNA from another species, i.e. not dogs, cats or humans. Advantageously, the use of at least one off-species control provides another layer of control to distinguish between background or non-specific signals and a positive result (for example, indicating the presence of disease in a subject).

Typically, the disease is selected from the group consisting of dilated cardiomyopathy and related conditions, valvular disease and related conditions, endocarditis, hypertrophic cardiomyopathy and related conditions, stenosis, atrial fibrillation and other rhythm disorders, cardiac tamponade/pericardial effusion, congenital disease and/or congestive heart failure, breed predispositions, parasitism, secondary conditions of other diseases, A/V node problems, toxic insults, dilation, hypertrophy and/or cardiovascular disease.

In one embodiment, the reference level may be provided by comparing the level of miRNA expression from the sample with an miRNA expression level from an unaffected control and a sample from a diseased animal.

Preferably, the sample is a biofluid selected from the group consisting of blood, urine, milk, tissue fluid, saliva, milk, cerebrospinal fluid (CSF) or another biofluid.

Preferably, the miRNAs are cell free miRNAs.

Advantageously, the method allows for high throughput, low cost testing that can be carried out and completed in a reasonable timeframe.

It is an advantage of the invention that the method can be used to accurately identify cardiovascular or heart disease in a subject using a sample of biofluid, such as a blood sample. Advantageously, the method allows for the identification of disease in an individual at an early stage and has the potential to transform patient care, quality of life and life expectancy. Advantageously, the miRNA profiles can allow heart damage to be detected at an early stage before any physical effects, structural changes and/or functional changes in the heart are detected.

According to a second aspect, there is provided a kit for use in performing the method of the first aspect comprising means for determining the level of expression of each one of the following miRNA molecules: cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p and hsa-miR-486-5p.

According to a third aspect, there is provided a method of selecting a panel for use in disease diagnosis comprising the steps of:
(a) selecting a group of miRNA molecules the differential expression of which may be associated with a disease condition;
(b) training at least one AI model to be able to predict the disease condition; and
(c) using the at least one AI model to reduce the number of miRNAs in the panel to a minimum number to provide a panel of miRNAs that still produces a result.

Preferably, the group of miRNA molecules comprise cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p and hsa-miR-486-5p.

The invention will now be described by way of example and with reference to the following Figures, wherein:

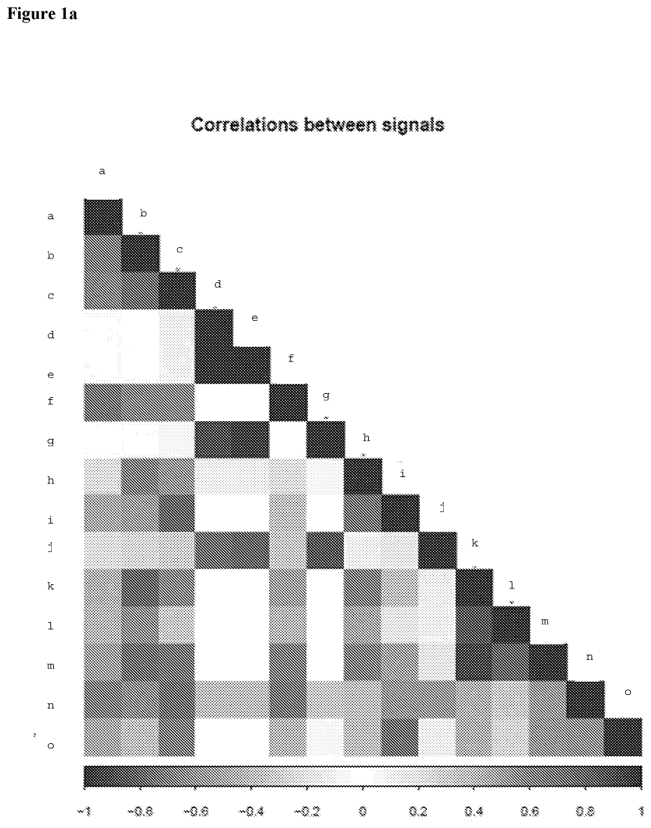

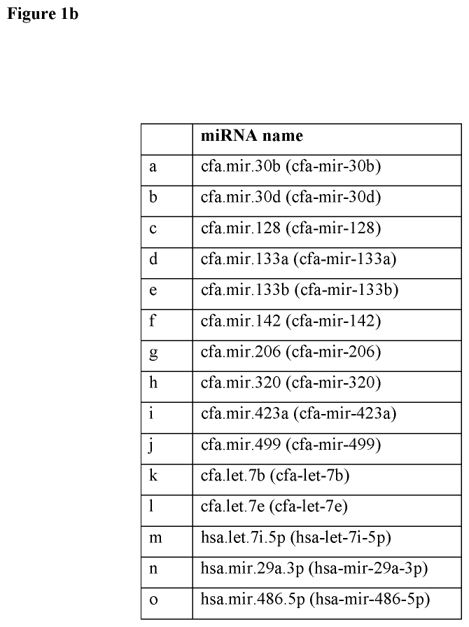

FIG. 1a is a chart showing the correlations that were found between pairs of signals;

FIG. 1b shows the names of the miRNA molecules used in FIG. 1a;

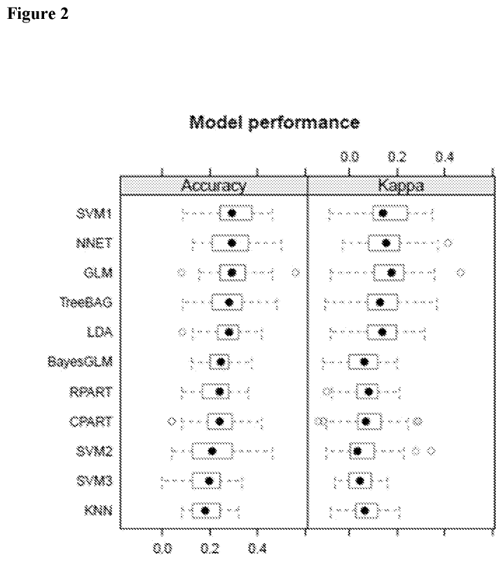

FIG. 2 shows a comparison of the machine learning models that were used to predict disease outcome from Example 1;

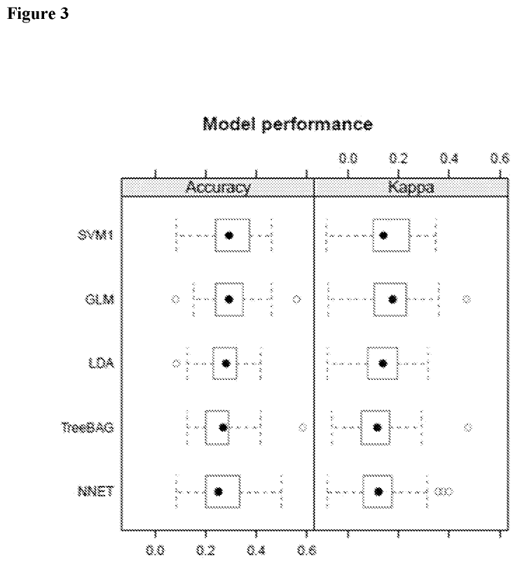

FIG. 3 shows a comparison of five machine learning models that were used to predict disease outcome from Example 1;

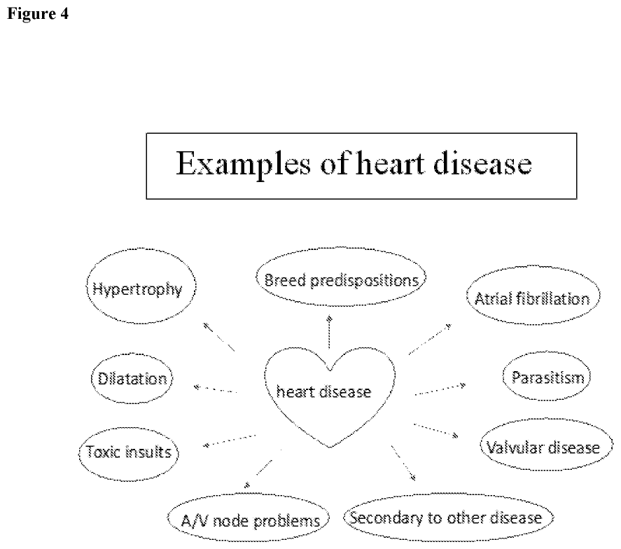

FIG. 4 shows examples of heart disease that may be present in a subject;

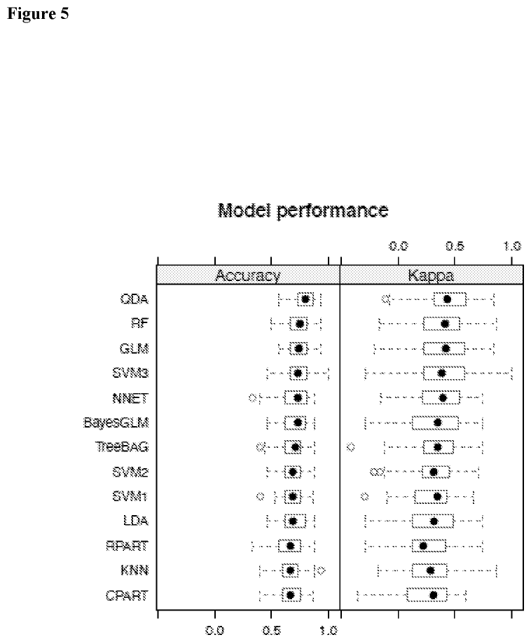

FIG. 5 shows a comparison of machine learning model performance using boxplots to represent the performance and variability throughout cross-validated data sets from canine samples from Example 1;

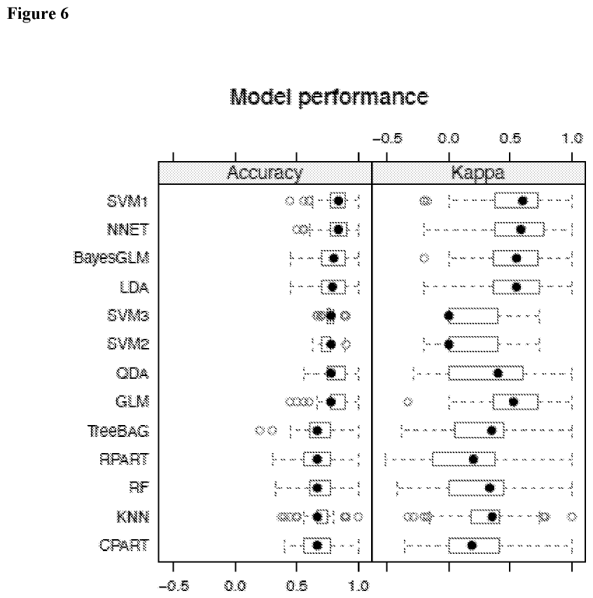

FIG. 6 shows a comparison of machine learning model performance using boxplots to represent the performance and variability throughout cross-validated data sets from canine samples from Example 1;

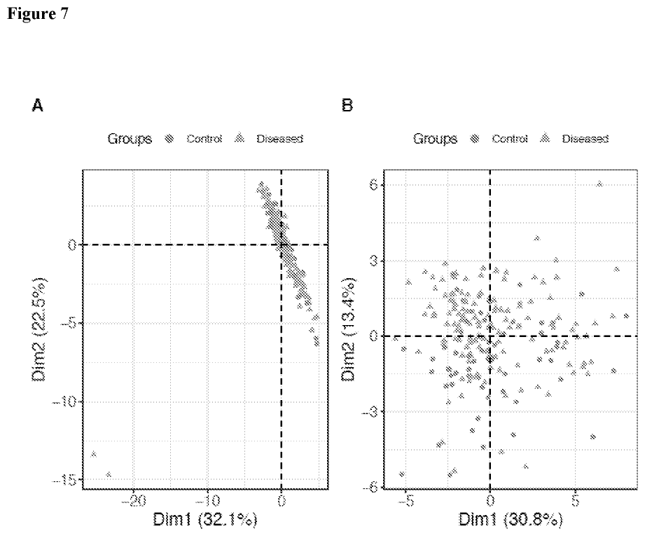

FIGS. 7a and 7b are PCA scores plots showing the results of the PCA analysis obtained during Example 2;

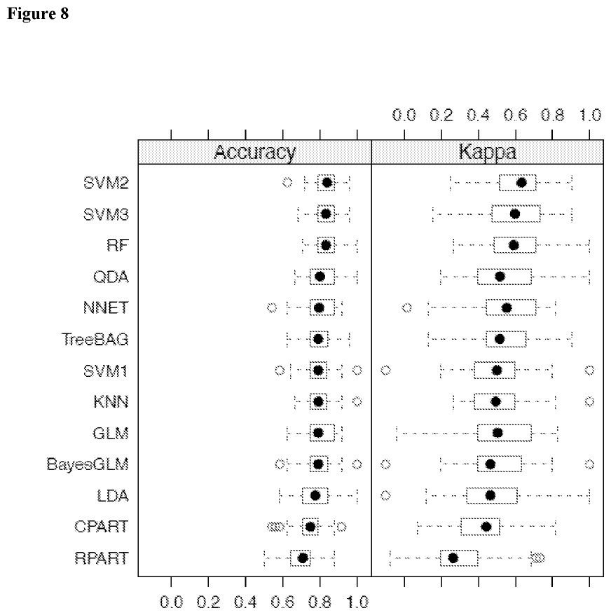

FIG. 8 shows a comparison of model performance for Example 2;

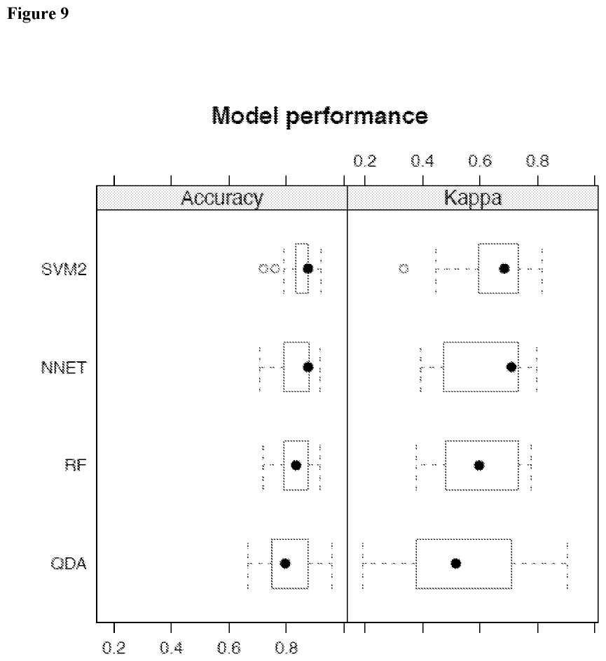

FIG. 9 shows a comparison of four machine learning models that were used to predict disease outcome from Example 2; and

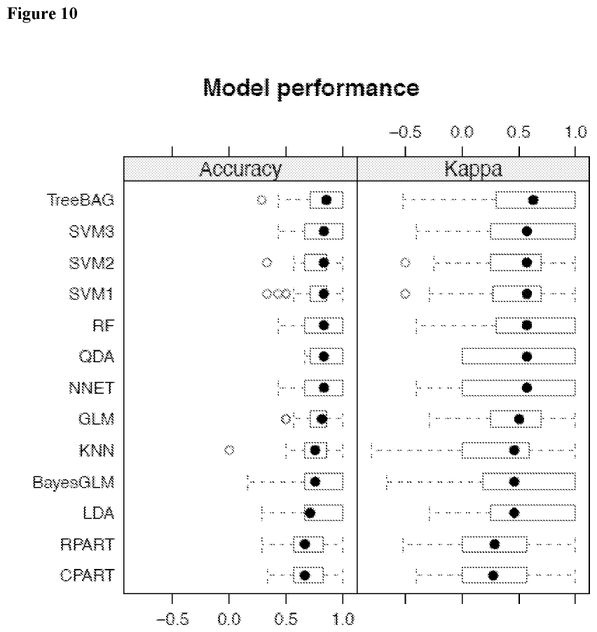

FIG. 10 shows a comparison of machine learning model performance using boxplots to represent the performance and variability throughout cross-validated data sets from feline samples from Example 2.

With reference to the figures, there is provided a method for detecting the presence of heart disease in a subject, comprising the steps of:
(a) determining the level of expression of each of a plurality of miRNAs within a sample from a subject; and
(b) using one or more Artificial Intelligence (AI) model to predict the disease condition of the subject.

The plurality of miRNAs form a panel comprising the following miRNA molecules: cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p, hsa-miR-486-5p.

The names of the miRNA molecules and associated sequences that are used in the method of the invention are set out below in Table 1.

TABLE 1

| miRNA | SEQ ID | |
|---|---|---|
| Name | Number | Sequence |
| cfa- | SEQ ID NO: 1 | UGUAAACAUCCUACACUCAGCU |
| miR- | | |
| 30b | | |
| cfa- | SEQ ID NO: | UGUAAACAUCCCCGACUGGAAGCU |
| miR- | 2 | |
| 30d | | |
| cfa- | SEQ ID NO: 3 | UCACAGUGAACCGGUCUCUUU |
| miR- | | |
| 128 | | |
| cfa- | SEQ ID NO: 4 | UUGGUCCCCUUCAACCAGCUGU |
| miR- | | |
| 133a | | |
| cfa- | SEQ ID NO: 5 | UUUGGUCCCCUUCAACCAGCUA |
| miR- | | |
| 133b | | |
| cfa- | SEQ ID NO: 6 | CCCAUAAAGUAGAAAGCACUA |
| miR- | | |
| 142 | | |
| cfa- | SEQ ID NO: 7 | UGGAAUGUAAGGAAGUGUGUGG |
| miR- | | |
| 206 | | |
| cfa- | SEQ ID NO: 8 | AAAAGCUGGGUUGAGAGGGCGA |
| miR- | | |
| 320 | | |
| cfa- | SEQ ID NO: | UGAGGGGCAGAGAGCGAGACUUU |
| miR- | 9 | |
| 423a | | |
| cfa- | SEQ ID NO: | UUAAGACUUGCAGUGAUGUUU |
| miR- | 10 | |
| 499 | | |
| cfa-let- | SEQ ID NO: | UGAGGUAGUAGGUUGUGUGGUU |
| 7b | 11 | |
| cfa-let- | SEQ ID NO: | UGAGGUAGGAGGUUGUAUAGUU |
| 7e | 12 | |
| hsa-let- | SEQ ID NO: | UGAGGUAGUAGUUUGUGCUGUU |
| 7i-5p | 13 | |
| hsa- | SEQ ID NO: | UAGCACCAUCUGAAAUCGGUUA |
| miR- | 14 | |
| 29a-3p | | |
| hsa- | SEQ ID NO: | UCCUGUACUGAGCUGCCCCGAG |
| miR- | 15 | |
| 486-5p | | |

The method further comprises the use of at least one normaliser and/or an off-species control miRNA molecule. At least one normaliser is used to ‘normalise’ data, i.e. to control for variation between the samples tested in the method of the invention, and the at least one control is used to try to ensure there are no failure or false readings in the results. An off-species control is added in to show that the miRNAs detected are relevant to the dog and/or cat panel. The off-species control is an miRNA from another species, i.e. not dogs, cats or humans. Advantageously, the use of an off-species controls provides another layer of control to distinguish between background or non-specific signals and a positive result.

The sequences of the normalisers and the off-species controls that were used are provided below in Table 2.

TABLE 2

| | SEQ ID | |
|---|---|---|
| | Number | Sequence |
| Normalizers | | |
| hsa-miR- | 16 | CAAAGUGCUUACAGUGCAGGUAG |
| 17-5p | | |
| cfa-miR- | 17 | CAGUGCAAUGAUGAAAGGGCAU |
| 130b | | |
| cfa-miR- | 18 | UAAAGUGCUUAUAGUGCAGGUAG |
| 20a | | |
| cfa-miR- | 19 | AUCACAUUGCCAGGGAUUU |
| 23a | | |
| cfa-miR-26a | 20 | UUCAAGUAAUCCAGGAUAGGCU |
| Off-species | | |
| controls | | |
| oan-miR- | 21 | UUCCCCACUCUGAGCACACAGC |
| 7417-5p | | |
| cel-mir-70- | 22 | UAAUACGUCGUUGGUGUUUCCAU |
| 3p | | |
| ath-mir167d | 23 | UGAAGCUGCCAGCAUGAUCUGG |

It is preferred that the method comprises the step of assessing the relative levels of miRNA expression of each one of miRNA molecules cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p, hsa-miR-486-5p within a sample from a subject and using the data obtained from measurement of the expression levels to determine the presence or absence of disease in a subject.

The disease is selected from the group consisting of cardiovascular disease, dilated cardiomyopathy and related conditions, valvular disease and related conditions, endocarditis, hypertrophic cardiomyopathy and related conditions, stenosis, atrial fibrillation and other rhythm disorders, cardiac tamponade/pericardial effusion, congenital disease and/or congestive heart failure. For example, the disease may be selected from the group of diseases shown in FIG. 4.

The sample is a biofluid selected from the group consisting of blood, urine, milk, tissue fluid, saliva, milk, cerebrospinal fluid (CSF) or another biofluid.

From the results of the above experiments, a differentiation in expression levels of miRNA was identified when comparing healthy dogs and cats with dogs and cats that have heart disease.

With reference to the figures, there is also provided a kit for use in performing the method of the first aspect comprising means for determining the level of expression of each one of the following miRNA molecules: cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p and hsa-miR-486-5p.

With reference to the figures, there is also provided a method of selecting a panel for use in disease diagnosis comprising the steps of:
(a) selecting a group of miRNA molecules the differential expression of which may be associated with a disease condition;
(b) training one or more AI model to be able to predict the disease condition; and
(c) using the one or more AI model to reduce the number of miRNAs in the panel to a minimum number to provide a panel of miRNAs that still produces a result.

There is therefore provided an miRNA assay to accurately identify the presence or absence of cardiovascular or heart disease in dogs and cats using a biofluid such as a blood sample. The method of the invention advantageously allows for the identification of disease at an early stage and has the potential to transform patient care, quality of life and life expectancy. Thus, the method, miRNAs and panel of the present invention can provide useful prognostic indicators for clinicians for patient monitoring and informed therapeutic intervention.

### Example 1

Samples were obtained from diseased and healthy cats and dogs. Diseased animals were selected on the basis of their disease morphology.

A particle mixture was added to each well of a 96 well microtitre plate. The particle mixture contained around 20 particles that are specific for miRNA molecules. The particle mixture was suspended in 10 μl biofluid taken from cat or dog subjects. In this case, the biofluid was blood. The particles were passed through a flow cytometer and around 20 readings were obtained for each of the 15 miRNA molecules from Table 1, with a maximum of 1400 data points per well.

The above method was carried out using FirePlex® Particle Technology (Abcam). FirePlex® Particle Technology uses FirePlex® particles (Abcam) which are made from a porous bio-inert hydrogel that allows targets to be captured throughout a 3D volume.

The FirePlex® assay protocol that was used in this example can be found in the FirePlex® miRNA Assay V3-Assay Protocol (Protocol Booklet Version 2.0, September 2018), which can also be found at the following link: https://www.abcam.com/ps/products/218/ab218370/documents/FirePlex %20miRNA %20Assay %20Protocol %20Booklet %20V-3a %20Dec%202018%20(website).pdf

The FirePlex® particles contain three distinct functional regions that are separated from each other by inert spacer regions. The central region of each particle is known as a central analyte or miRNA quantification region which contains miRNA probes that can capture target miRNAs. The central region of the particle comprises a reporter dye. The two end regions of each particle act as two halves of a barcode that distinguish between different particles. Detection is carried out using a flow cytometer to detect miRNA molecules that emit fluorescence that is proportional to their abundance in the sample. The flow cytometer was used to detect the fluorescence signal from the centre of each particle through the reporter dye. Each miRNA that was used was given a unique code (up to 70 different codes were possible). The data that was obtained from the mixture of particles could then be attributed to the miRNAs by identification of the code.

After the data acquisition, software called FirePlex® Analysis Workbench software was used to merge the events that were obtained from the three regions of the particles into a single event. Abundance data was then obtained for each miRNA molecule.

The data set for this experiment included 248 miRNA samples (including 156 canine samples and 92 feline samples). The data set included 178 diseased and 70 control samples.

An example of the data obtained from the above experiment is provided below in Table 3. As mentioned above, the data set included 248 miRNA samples. The results below are shown for one of the diseased samples and one of the control samples used in this experiment. Data was collected for each of the 15 miRNA samples mentioned in Table 1. The results obtained with the normalisers as mentioned in Table 2 are also shown.

TABLE 3

| | | cfa-mir- | cfa-mir- | cfa-mir- | cfa-mir- | cfa-mir- | cfa-mir- | cfa-mir- | cfa-mir- |
|---|---|---|---|---|---|---|---|---|---|
| Species | Diagnosis | 30b | 30d | 128 | 133a | 133b | 142 | 206 | 320 |
| Canine | diseased | 438.479 | 58.336 | 452.258 | 0.819 | −0.587 | 70.898 | 0.37 | 1180.699 |
| Canine | control | 326.123 | 67.46 | 203.404 | 11.962 | 4.074 | 146.065 | 3.146 | 700.702 |

| | | cfa-mir- | cfa-mir- | cfa-let- | cfa-let- | hsa-let- | hsa-mir- | hsa-mir- |
|---|---|---|---|---|---|---|---|---|
| Species | Diagnosis | 423a | 499 | 7b | 7e | 7i-5p | 29a-3p | 486-5p |
| Canine | diseased | 2433.454 | 2.778 | 210.883 | 5.179 | 221.919 | 317.943 | 3483.807 |
| Canine | control | 1299.002 | 14.349 | 279.72 | 7.068 | 400.665 | 426.017 | 5852.449 |

| | | hsa-mir-17-5p | cfa-mir-130b | cfa-mir-20a | cfa-mir-23a | cfa-mir-26a |
|---|---|---|---|---|---|---|
| Species | Diagnosis | normaliser | normaliser | normaliser | normaliser | normaliser |
| Canine | diseased | 1556.018 | 386.968 | 926.496 | 462.396 | 40.9 |
| Canine | control | 748.865 | 64.225 | 856.749 | 421.9 | 81.113 |

Along with the above, pre-processed miRNA profiles consisting of signals were provided for each sample. The objective was to build a predictive model of disease outcome based on the miRNA signals.

Exploratory Data Analysis

Exploratory Data Analysis was carried out to examine data and look for trends of the results following the FirePlex® analysis.

FIG. 1a summarises the correlations between pairs of signals. They are generally positive and moderate. Signals cfa.mir.133a (i.e. cfa-mir-133a) and cfa.mir.133b (i.e. cfa-mir-133b) appear to be strongly correlated between them (r=0.98) and with cfa.mir.206 (r=0.90 and r=0.95 correlation with cfa.mir.133a and cfa.mir.133b respectively), but weakly correlated with most of the others.

Principal component analysis (PCA) was used to compute new variables (the principal components; PCs) which are uncorrelated linear combinations of the miRNA signals. By comparison, successive principal components summarise decreasing portions of the total variability in the original data. In particular, the two first PCs account for the highest portion and are used to approximately represent the data in a 2D graph called a biplot. A biplot jointly represents both samples and miRNA signals, using point and rays, respectively.

The proximity between points relates to the similarity between samples according to their miRNA profiles. The rays indicate directions of increasing intensity of the signals, whereas the angles between the rays are related to the correlations between them: the smaller the angle the higher the positive correlation, the closer to right angle the weaker the correlation, and the closer to straight angle the higher the negative correlation. Hence, for the present purposes, a PCA biplot facilitates the visualisation and identification of patterns in the data.

The Exploratory Data Analysis was carried out for information purposes, e.g. to understand any trends that were seen in the data.

Some pre-processing was conducted to impute a few missing signals for some samples. The signals were log-transformed for improved visualisation.

Predictive Modelling

The objective of the predictive modelling was to investigate the scope to use the miRNA profiles to predict the presence or absence of disease.

A group of healthy and unhealthy animals were taken and tested to determine the level of miRNA expression in samples from these animals. The data obtained was then used to train the models.

Eleven machine learning models were fitted and compared with the aim of obtaining the best predictions of the disease outcome. An important consideration in respect of the data set for this example was the relatively large difference between the number of samples belonging to the different disease outcomes. In this case, a sampling procedure called SMOTE was used with the aim to correct for this unbalanced class problem while comparing the performance of the models. A number of statistics based on 5-time repeated 10-fold cross-validation were calculated for each model. Cross-validation was useful to obtain more realistic model performance measures from the training data.

Data from the FirePlex® analysis from each of the fifteen miRNA molecules from Table 1 was fitted to each of the models.

The following summary statistics shown in Table 4 and FIG. 2 compare model performance in terms of accuracy (proportion of samples for which the model predicted the right outcome) and the Kappa metric (values between 0 and 1) indicates how good the model of prediction is in relation to simply allocating samples to classes at random. In the graph shown in FIG. 2, the models are ordered from best (top) to worst (bottom) relative performance using boxplots to represent the performance throughout cross-validated data sets. The black dot indicates the median estimate and the whiskers the most extreme estimates.

TABLE 4
Call:
Summary.resamples (object = resampsSMOTE)
Models: CPART, GLM, LDA, BayesGLM, KNN,
NNET, SVM1, SVM2, SVM3, RPART, TreeBAG
Number of resamples: 50

| | | | | | | | |
|---|---|---|---|---|---|---|---|
| Model | Min | 1^{st} Qu | Median | Mean | 3^{rd} Qu | Max | NA's |

Accuracy

| | | | | | | | |
|---|---|---|---|---|---|---|---|
| CPART | 0.0385 | 0.192 | 0.240 | 0.239 | 0.292 | 0.417 | 0 |
| GLM | 0.0800 | 0.240 | 0.292 | 0.299 | 0.343 | 0.560 | 0 |
| LDA | 0.0833 | 0.233 | 0.280 | 0.273 | 0.320 | 0.417 | 0 |
| BayesGLM | 0.1200 | 0.200 | 0.245 | 0.241 | 0.280 | 0.375 | 0 |
| KNN | 0.0800 | 0.132 | 0.179 | 0.186 | 0.238 | 0.320 | 8 |
| NNET | 0.1250 | 0.208 | 0.292 | 0.290 | 0.353 | 0.500 | 0 |
| SVM1 | 0.0833 | 0.240 | 0.292 | 0.297 | 0.371 | 0.462 | 0 |
| SVM2 | 0.0400 | 0.125 | 0.208 | 0.205 | 0.289 | 0.462 | 0 |
| SVM3 | 0.0000 | 0.132 | 0.196 | 0.182 | 0.240 | 0.333 | 0 |
| RPART | 0.0800 | 0.167 | 0.240 | 0.225 | 0.277 | 0.360 | 0 |
| TreeBAG | 0.0833 | 0.208 | 0.280 | 0.272 | 0.330 | 0.480 | 0 |

Kappa

| | | | | | | | |
|---|---|---|---|---|---|---|---|
| CPART | −0.1304 | 0.035408 | 0.0680 | 0.0826 | 0.129 | 0.290 | 0 |
| GLM | −0.0788 | 0.102503 | 0.1757 | 0.1708 | 0.225 | 0.467 | 0 |
| LDA | −0.0820 | 0.080660 | 0.1368 | 0.1352 | 0.194 | 0.314 | 0 |
| BayesGLM | −0.1111 | 0.004839 | 0.0610 | 0.0608 | 0.117 | 0.202 | 0 |
| KNN | −0.0798 | 0.026073 | 0.0634 | 0.0670 | 0.115 | 0.211 | 8 |
| NNET | −0.0288 | 0.080686 | 0.1531 | 0.1501 | 0.206 | 0.413 | 0 |
| SVM1 | −0.0864 | 0.100000 | 0.1395 | 0.1547 | 0.241 | 0.346 | 0 |
| SVM2 | −0.0980 | 0.003271 | 0.0323 | 0.0590 | 0.101 | 0.343 | 0 |
| SVM3 | −0.0629 | 0.000434 | 0.0429 | 0.0447 | 0.087 | 0.159 | 0 |
| RPART | −0.0978 | 0.031729 | 0.0796 | 0.0706 | 0.116 | 0.211 | 0 |
| TreeBAG | −0.1046 | 0.077562 | 0.1271 | 0.1318 | 0.201 | 0.365 | 0 |

From the data above it can be seen that there are not large differences between models.

FIG. 3 focusses on the top five models. It should be noted that the boxplots shown in FIG. 3 are not exactly the same as those shown in FIG. 2 because a different random seed was used to generate the cross-validation sets (although these were the same for all models in each comparison). The statistics of the top five models are set out below in Table 5:

TABLE 5
Call:
Summary.resamples (object = resampsSMOTEtop)
Models: SVM1, NNET, GLM, TreeBAG, LDA
Number of resamples: 50

| | | | | | | | |
|---|---|---|---|---|---|---|---|
| Model | Min | 1^{st} Qu | Median | Mean | 3^{rd} Qu | Max | NA's |

Accuracy

| | | | | | | | |
|---|---|---|---|---|---|---|---|
| SSVM1 | 0.0833 | 0.240 | 0.292 | 0.297 | 0.371 | 0.462 | 0 |
| NNET | 0.0833 | 0.200 | 0.250 | 0.270 | 0.333 | 0.500 | 0 |
| GLM | 0.0800 | 0.240 | 0.292 | 0.299 | 0.343 | 0.560 | 0 |
| TreeBAG | 0.1250 | 0.200 | 0.269 | 0.259 | 0.292 | 0.583 | 0 |
| LDA | 0.0833 | 0.233 | 0.280 | 0.273 | 0.320 | 0.417 | 0 |

Kappa

| | | | | | | | |
|---|---|---|---|---|---|---|---|
| SSVM1 | −0.0864 | 0.1000 | 0.139 | 0.155 | 0.241 | 0.346 | 0 |
| NNET | −0.0827 | 0.0587 | 0.120 | 0.133 | 0.173 | 0.397 | 0 |
| GLM | −0.0788 | 0.1025 | 0.176 | 0.171 | 0.225 | 0.467 | 0 |
| TreeBAG | −0.0655 | 0.0538 | 0.115 | 0.115 | 0.163 | 0.474 | 0 |
| LDA | −0.0820 | 0.0807 | 0.137 | 0.135 | 0.194 | 0.314 | 0 |

From the above, it can be seen that the results are very much comparable between the models.

The above experiment was run to see if it was possible to distinguish between different disease classes. On the basis of the results, the accuracy in this case was approximately 30%.

Canine Species

Table 6 below summarises the canine samples by category. It shows a large difference between the number of diseased and control samples that were available.

TABLE 6
Disease class frequencies:

| Control | Diseased |
|---|---|
| 46 | 110 |

Predictive models were fitted using the miRNA profiles as predictors of disease outcome. The following summary statistics shown in Table 7 and FIG. 5 compare model performance in terms of accuracy (proportion of samples for which the model predicted the right outcome) and the Kappa metric (values between 0 and 1, indicates how good the prediction is in relation to simply allocating samples to classes at random). In FIG. 5, the models are ordered from best (top) to worst (bottom) relative performance using boxplots to represent the performance and variability throughout cross-validated data sets. The black dot indicates the median estimate and the whiskers the most extreme estimates. The main statistics used for performance assessment is the mean value.

TABLE 7
Call:
summary.resamples (object = resampsSMOTE)
Models: CPART, GLM, LDA, Bayes GLM, KNN, NNET,
QDA, SVM1, SVM2, SVM3, RF, RPART, TreeBAG
Number of resamples: 50

| | | | | | | | |
|---|---|---|---|---|---|---|---|
| Model | Min | 1^{st} Qu | Median | Mean | 3^{rd} Qu | Max | NA's |

Accuracy

| | | | | | | | |
|---|---|---|---|---|---|---|---|
| CPART | 0.400 | 0.600 | 0.667 | 0.664 | 0.750 | 0.867 | 0 |
| GLM | 0.562 | 0.667 | 0.742 | 0.738 | 0.812 | 0.938 | 0 |
| LDA | 0.467 | 0.625 | 0.688 | 0.697 | 0.800 | 0.875 | 0 |
| BayesGLM | 0.467 | 0.625 | 0.733 | 0.702 | 0.800 | 0.875 | 0 |
| KNN | 0.400 | 0.600 | 0.667 | 0.661 | 0.733 | 0.938 | 0 |
| NNET | 0.333 | 0.625 | 0.733 | 0.700 | 0.809 | 0.875 | 0 |
| QDA | 0.562 | 0.733 | 0.800 | 0.786 | 0.853 | 0.938 | 0 |
| SVM1 | 0.400 | 0.625 | 0.688 | 0.687 | 0.750 | 0.867 | 0 |
| SVM2 | 0.467 | 0.635 | 0.688 | 0.705 | 0.750 | 0.875 | 0 |
| SVM3 | 0.467 | 0.667 | 0.733 | 0.723 | 0.812 | 1.000 | 0 |
| RF | 0.500 | 0.667 | 0.750 | 0.734 | 0.809 | 0.938 | 0 |
| RPART | 0.333 | 0.572 | 0.667 | 0.654 | 0.746 | 0.875 | 0 |
| TreeBAG | 0.400 | 0.635 | 0.710 | 0.698 | 0.750 | 0.875 | 0 |

Kappa

| | | | | | | | |
|---|---|---|---|---|---|---|---|
| CPART | −0.364 | 0.0748 | 0.310 | 0.263 | 0.426 | 0.595 | 0 |
| GLM | −0.216 | 0.2241 | 0.418 | 0.398 | 0.586 | 0.846 | 0 |
| LDA | −0.296 | 0.1320 | 0.314 | 0.308 | 0.478 | 0.738 | 0 |
| BayesGLM | −0.296 | 0.1320 | 0.347 | 0.322 | 0.526 | 0.738 | 0 |
| KNN | −0.176 | 0.1256 | 0.284 | 0.288 | 0.424 | 0.862 | 0 |
| NNET | −0.154 | 0.2112 | 0.393 | 0.355 | 0.534 | 0.738 | 0 |
| QDA | −0.116 | 0.3182 | 0.431 | 0.436 | 0.593 | 0.846 | 0 |
| SVM1 | −0.296 | 0.1630 | 0.345 | 0.311 | 0.429 | 0.659 | 0 |
| SVM2 | −0.216 | 0.2105 | 0.312 | 0.298 | 0.438 | 0.709 | 0 |
| SVM3 | −0.296 | 0.2258 | 0.383 | 0.396 | 0.586 | 1.000 | 0 |
| RF | −0.164 | 0.2258 | 0.412 | 0.390 | 0.538 | 0.862 | 0 |
| RPART | −0.296 | 0.1233 | 0.219 | 0.235 | 0.411 | 0.738 | 0 |
| TreeBAG | −0.421 | 0.2258 | 0.347 | 0.337 | 0.473 | 0.738 | 0 |

From the above, it can be seen that there were not large differences between models. The best accuracies were around 80% in mean and the best Kappa metrics are around 40%. The results below show for the top model (QBA) the so-called confusion matrix confronting predicted versus observed outcomes across cross-validation resamples. The values are proportions for each actual predicted combination across resamples. Errors for each class are off the diagonal (about 14.23% of control samples were wrongly classified as diseased samples and about 7.18% of the diseased samples were wrongly classified as control samples). Afterwards, a number of model performance statistics are provided, including overall mean accuracy (78.6%), a 95% confidence interval for this, and sensitivity (89.8%) and specificity (51.7%) amongst others, with the diseased class corresponding to the positive outcome of the test.

The statistics are shown below in Table 8.

TABLE 8
Confusion Matrix and Statistics
Reference

| Predication | Diseased | Control |
|---|---|---|
| Diseased | 0.6333 | 0.1423 |
| Control | 0.0718 | 0.1526 |
| | Accuracy: | 0.786 |
| | 95% CI: | (0.755, 0.814) |

| | |
|---|---|
| No Information Rate: | 0.705 |
| P-Value [Acc > NIR]: | 2.15e−07 |
| Kappa: | 0.447 |
| Mcnemar's Test P-Value: | 2.93e−05 |
| Sensitivity: | 0.898 |
| Specificity: | 0.517 |
| Pos Pred Value: | 0.817 |
| Neg Pred Value: | 0.680 |
| Prevalence: | 0.705 |
| Detection Rate: | 0.633 |
| Detection Prevalence: | 0.776 |
| Balanced Accuracy: | 0.708 |
| ‘Positive’ Class: | Diseased |

Thus, it can be seen that the accuracy of this experiment above was improved to 80%. This improvement was due to the fact that the AI models were assessing the presence or absence of disease in a subject. Thus, when using the method to determine the presence or absence of disease in a subject, the accuracy was high, i.e. approximately 80%.

Feline Species

The same analysis was conducted using the feline samples. Table 9 shows a large difference between the number of diseased and control samples available.

TABLE 9
Disease class frequencies:

| Control | Diseased |
|---|---|
| 24 | 68 |

As above, the data below in Table 10 and FIG. 6 compare the corresponding models in terms of accuracy and Kappa metric.

TABLE 10
Call:
summary.resamples (object = resampsSMOTE)
Models: CPART, GLM, LDA, Bayes GLM, KNN, NNET,
QDA, SVM1, SVM2, SVM3, RF, RPART, TreeBAG
Number of resamples: 50

| | | | | | | | |
|---|---|---|---|---|---|---|---|
| Model | Min | 1^{st} Qu | Median | Mean | 3^{rd} Qu | Max | NA's |

Accuracy

| | | | | | | | |
|---|---|---|---|---|---|---|---|
| CPART | 0.400 | 0.557 | 0.667 | 0.678 | 0.778 | 1.0 | 0 |
| GLM | 0.444 | 0.778 | 0.778 | 0.809 | 0.889 | 1.0 | 0 |
| LDA | 0.444 | 0.700 | 0.789 | 0.807 | 0.889 | 1.0 | 0 |
| BayesGLM | 0.444 | 0.712 | 0.800 | 0.811 | 0.889 | 1.0 | 0 |
| KNN | 0.375 | 0.667 | 0.667 | 0.684 | 0.750 | 1.0 | 0 |
| NNET | 0.500 | 0.778 | 0.838 | 0.821 | 0.900 | 1.0 | 0 |
| QDA | 0.556 | 0.750 | 0.778 | 0.787 | 0.889 | 1.0 | 0 |
| SVM1 | 0.444 | 0.778 | 0.838 | 0.821 | 0.889 | 1.0 | 0 |
| SVM2 | 0.625 | 0.712 | 0.778 | 0.768 | 0.778 | 0.9 | 0 |
| SVM3 | 0.667 | 0.750 | 0.778 | 0.770 | 0.778 | 0.9 | 0 |
| RF | 0.333 | 0.600 | 0.667 | 0.684 | 0.778 | 1.0 | 0 |
| RPART | 0.300 | 0.556 | 0.667 | 0.661 | 0.778 | 1.0 | 0 |
| TreeBAG | 0.200 | 0.600 | 0.667 | 0.675 | 0.778 | 1.0 | 0 |

Kappa

| | | | | | | | |
|---|---|---|---|---|---|---|---|
| CPART | −0.364 | 0.0119 | 0.188 | 0.233 | 0.412 | 1.000 | 0 |
| GLM | −0.333 | 0.3571 | 0.526 | 0.533 | 0.727 | 1.000 | 0 |
| LDA | −0.200 | 0.3571 | 0.549 | 0.535 | 0.734 | 1.000 | 0 |
| BayesGLM | −0.200 | 0.3571 | 0.549 | 0.538 | 0.727 | 1.000 | 0 |
| KNN | −0.333 | 0.1818 | 0.352 | 0.305 | 0.409 | 1.000 | 0 |
| NNET | −0.200 | 0.3721 | 0.586 | 0.555 | 0.761 | 1.000 | 0 |
| QDA | −0.286 | 0.0000 | 0.400 | 0.278 | 0.609 | 1.000 | 0 |
| SVM1 | −0.200 | 0.3721 | 0.600 | 0.555 | 0.727 | 1.000 | 0 |
| SVM2 | −0.200 | 0.0000 | 0.000 | 0.140 | 0.389 | 0.737 | 0 |
| SVM3 | 0.000 | 0.0000 | 0.000 | 0.144 | 0.389 | 0.737 | 0 |
| RF | −0.421 | 0.0119 | 0.333 | 0.249 | 0.436 | 1.000 | 0 |
| RPART | −0.522 | −0.1084 | 0.200 | 0.205 | 0.372 | 1.000 | 0 |
| TreeBAG | −0.379 | 0.0489 | 0.348 | 0.254 | 0.426 | 1.000 | 0 |

From the above results, it can be seen that there are not large differences between models. The best accuracies are around 82% in mean and the best Kappa metrics are around 55%. The following table shows the so-called confusion matrix confronting predicted versus observed outcomes across cross-validation resamples for the best performing SVM1 model above. The values are proportions for each actual-predicted combination across resamples. Errors for each class are off the diagonal (about 6.09% of control samples were wrongly classified as diseased samples and about 11.52% of the diseased samples were wrongly classified as control samples). Afterwards, a number of model performance statistics are provided, including overall mean accuracy (82.4%), a 95% confidence interval for this, and sensitivity (84.4%) and specificity (76.7%) amongst others, with the diseased class corresponding to the positive outcome of the test. Thus, the results are similar to the ones based on canine samples, although with some better specificity in the feline case.

The statistics of the above results are shown below in Table 11.

TABLE 11
Confusion Matrix and Statistics
Reference

| Prediction | Diseased | Control |
|---|---|---|
| Diseased | 0.6239 | 0.0609 |
| Control | 0.1152 | 0.2000 |
| | Accuracy: | 0.824 |
| | 95% CI: | (0.786, 0.858) |

| | |
|---|---|
| No Information Rate: | 0.739 |
| P-Value [Acc > NIR]: | 1.07e−05 |
| Kappa: | 0.572 |
| Mcnemar's Test P-Value: | 0.00766 |
| Sensitivity: | 0.844 |
| Specificity: | 0.767 |
| Pos Pred Value: | 0.911 |
| Neg Pred Value: | 0.634 |
| Prevalence: | 0.739 |
| Detection Rate: | 0.624 |
| Detection Prevalence: | 0.685 |
| Balanced Accuracy: | 0.805 |
| ‘Positive’ Class: | Diseased |

### Example 2

Samples were obtained from diseased and healthy cats and dogs. Diseased animals were selected on the basis of their disease morphology.

In the following experiment, the data set included 309 miRNA samples (including 244 canine samples and 65 feline samples).

Using the FirePlex® technology as described in Example 1, a particle mixture was added to each well of a 96 well microtitre plate. The particle mixture contained around 20 particles specific for miRNA molecules. The particle mixture was suspended in 10 μl biofluid taken from canine and feline species. The particles were passed through a flow cytometer and around 20 readings were obtained for every miRNA molecule, with a maximum of 1400 data points per well.

An example of the data obtained from the above experiment is provided below in Table 12. As mentioned above, the data set included 248 miRNA samples. The results below are shown for one of the diseased samples and one of the control samples used in this experiment. Data was collected for each of the 15 miRNA samples mentioned in Table 1. The results obtained with the normalisers and controls as mentioned in Table 2 are also shown.

TABLE 12

| | | cfa-mir- | cfa-mir- | cfa-mir- | cfa-mir- | cfa-mir- | cfa-mir- | cfa-mir- |
|---|---|---|---|---|---|---|---|---|
| Species | Diagnosis | 30b | 30d | 128 | 133a | 133b | 142 | 206 |
| Canine | diseased | 7716.47 | 8912.39 | 25382.13 | 1370.33 | 1340.18 | 13371.43 | 1379.66 |
| Canine | control | 4791.38 | 4080.49 | 34663.49 | 1904.22 | 2161.21 | 10724.18 | 1850.56 |

| | | cfa-mir- | cfa-mir- | cfa-mir- | cfa-let- | cfa-let- | hsa-let- | hsa-mir- |
|---|---|---|---|---|---|---|---|---|
| Species | Diagnosis | 320 | 423a | 499 | 7b | 7e | 7i-5p | 29a-3p |
| Canine | diseased | 60507.20 | 121752.24 | 2634.55 | 29523.97 | 2753.65 | 31606.88 | 24992.33 |
| Canine | control | 134872.83 | 268417.84 | 1898.75 | 19339.97 | 3253.41 | 20673.67 | 52012.84 |

| | | hsa-mir- | hsa-mir- | cfa-mir- | cfa-mir- | cfa-mir- | cfa-mir- | oan-mir- |
|---|---|---|---|---|---|---|---|---|
| Species | Diagnosis | 486-5p | 17-5p | 130b | 20a | 23a | 26a | 7417-5p |
| Canine | diseased | 390438.9 | 54512.46 | 13573.62 | 55458.72 | 16775.10 | 2031.02 | 1248.98 |
| Canine | control | 879402.80 | 35355.76 | 12487.89 | 17537.84 | 35372.78 | 3166.31 | 1850.16 |

| | | cel-mir- | ath- |
|---|---|---|---|
| Species | Diagnosis | 70-3p | mir167d |
| Canine | diseased | 1292.86 | 1395.09 |
| Canine | control | 1720.56 | 1698.82 |

Canine Species

As in Example 1, an Exploratory Data Analysis was carried out as a first step to assess the data. A principal component analysis (PCA) provided a synthetic view of the data set. In particular, first two PCs were used, i.e. those accounting for the highest proportion of variability in the data set, to project the data into a 2-dimensional graphical representation to facilitate the investigation of relationships and patterns in the data. In this case, the miRNA signals were log-transformed for improved visualisation. FIGS. 7a and 7b show the PCA scores (representing the original samples in two dimensions; percentage variability explained by each PC is shown within parenthesis on the axis labels). Different symbols were used to distinguish the samples according to the presence or absence of disease. The means of each group (shown as bigger symbols) are relatively close to the origin of the plot (representing the overall means). The results shown in FIG. 7a show two outlying samples that were identified in the raw data. These samples were considered to be abnormal measurements and were therefore removed from subsequent analysis. FIG. 7b shows the PCA plot scores without the two abnormal samples from FIG. 7a.

As for Experiment 1, the Exploratory Data Analysis was used to look for trends and assess the data.

A group of healthy and unhealthy animals were taken and tested to determine the level of miRNA expression in samples from these animals. The data obtained was then used to train the models.

Predictive models were used to assess the miRNA profiles as predictors of disease outcome. The focus was on differentiating between diseased versus control cases. Given the large difference between the number of samples belonging to each group (72 control versus 172 diseased samples) a resampling procedure called SMOTE was used with aims to correct for the unbalanced classes problem while comparing the performance of the models. A number of statistics based on 5-time repeated 10-fold cross-validation were calculated for each model. Cross-validation is useful to obtain more realistic model performance measures from training data.

Data from the FirePlex® analysis using the 15 miRNA molecules from Table 1 was fitted with the models. The following summary statistics shown in Table 13 and FIG. 8 compare model performance in terms of accuracy (proportion of samples for which to model predicted the right outcome) and the Kappa metric (values between 0 and 1, indicate how good in the prediction in relation to simply allocating samples to classes at random). In the graph, the models are ordered from best (top) to worst (bottom) relative performance using boxplots to represent the performance and variability throughout cross-validated data sets. The black dot indicates the median estimate and the whiskers the most extreme estimates. The main statistic used for performance assessment is the mean value.

TABLE 13
Call:
summary.resamples (object = resampsSMOTE)
Models: CPART, GLM, LDA, Bayes GLM, KNN, NNET,
QDA, SVM1, SVM2, SVM3, RF, RPART, TreeBAG
Number of resamples: 50

| | | | | | | | |
|---|---|---|---|---|---|---|---|
| Model | Min | 1^{st} Qu | Median | Mean | 3^{rd} Qu | Max | NA's |

Accuracy

| | | | | | | | |
|---|---|---|---|---|---|---|---|
| CPART | 0.542 | 0.708 | 0.750 | 0.751 | 0.792 | 0.917 | 0 |
| GLM | 0.625 | 0.750 | 0.792 | 0.791 | 0.866 | 0.920 | 0 |
| LDA | 0.583 | 0.708 | 0.776 | 0.783 | 0.838 | 1.000 | 0 |
| BayesGLM | 0.583 | 0.750 | 0.792 | 0.784 | 0.840 | 1.000 | 0 |
| KNN | 0.667 | 0.750 | 0.792 | 0.792 | 0.833 | 1.000 | 0 |
| NNET | 0.542 | 0.750 | 0.796 | 0.801 | 0.875 | 0.920 | 0 |
| QDA | 0.667 | 0.752 | 0.800 | 0.820 | 0.875 | 1.000 | 0 |
| SVM1 | 0.583 | 0.750 | 0.792 | 0.786 | 0.833 | 1.000 | 0 |
| SVM2 | 0.625 | 0.792 | 0.840 | 0.837 | 0.875 | 0.958 | 0 |
| SVM3 | 0.680 | 0.792 | 0.833 | 0.834 | 0.879 | 0.958 | 0 |
| RF | 0.708 | 0.792 | 0.833 | 0.827 | 0.875 | 1.000 | 0 |
| RPART | 0.500 | 0.640 | 0.708 | 0.700 | 0.750 | 0.875 | 0 |
| TreeBAG | 0.625 | 0.750 | 0.792 | 0.795 | 0.838 | 0.958 | 0 |

Kappa

| | | | | | | | |
|---|---|---|---|---|---|---|---|
| CPART | 0.0698 | 0.310 | 0.442 | 0.430 | 0.517 | 0.814 | 0 |
| GLM | −0.0385 | 0.400 | 0.503 | 0.511 | 0.677 | 0.828 | 0 |
| LDA | −0.1009 | 0.336 | 0.464 | 0.485 | 0.604 | 1.000 | 0 |
| BayesGLM | −0.1009 | 0.395 | 0.464 | 0.494 | 0.623 | 1.000 | 0 |
| KNN | 0.2632 | 0.382 | 0.493 | 0.518 | 0.597 | 1.000 | 0 |
| NNET | 0.0149 | 0.442 | 0.552 | 0.547 | 0.710 | 0.816 | 0 |
| QDA | 0.1923 | 0.395 | 0.516 | 0.541 | 0.684 | 1.000 | 0 |
| SVM1 | −0.1009 | 0.382 | 0.499 | 0.493 | 0.597 | 1.000 | 0 |
| SVM2 | 0.2500 | 0.516 | 0.632 | 0.610 | 0.710 | 0.903 | 0 |
| SVM3 | 0.1525 | 0.484 | 0.597 | 0.608 | 0.731 | 0.903 | 0 |
| RF | 0.2632 | 0.482 | 0.590 | 0.597 | 0.710 | 1.000 | 0 |
| RPART | −0.0787 | 0.192 | 0.263 | 0.279 | 0.391 | 0.731 | 0 |
| TreeBAG | 0.1290 | 0.442 | 0.515 | 0.540 | 0.648 | 0.903 | 0 |

From the data, it can be seen that there were not large differences between models. The best accuracies were around 80% and the best Kappa metrics were around 60%. FIG. 9 and the data below in Table 14 focuses on the top four models. These new boxplots are not exactly the same as those shown above because a different random seed was used to generate the cross-validation sets.

TABLE 14
Call:
summary.resamples (object = resampsSMOTE)
Models: SVM2, RF, QDA, NNET
Number of resamples: 14

| | | | | | | | |
|---|---|---|---|---|---|---|---|
| Model | Min | 1^{st} Qu | Median | Mean | 3^{rd} Qu | Max | NA's |

Accuracy

| | | | | | | | |
|---|---|---|---|---|---|---|---|
| SVM2 | 0.720 | 0.833 | 0.875 | 0.850 | 0.875 | 0.920 | 0 |
| RF | 0.720 | 0.792 | 0.833 | 0.826 | 0.875 | 0.917 | 0 |
| QDA | 0.667 | 0.760 | 0.796 | 0.809 | 0.865 | 0.958 | 0 |
| NNET | 0.708 | 0.792 | 0.875 | 0.834 | 0.879 | 0.917 | 0 |

Kappa

| | | | | | | | |
|---|---|---|---|---|---|---|---|
| SVM2 | 0.335 | 0.597 | 0.684 | 0.646 | 0.726 | 0.816 | 0 |
| RF | 0.377 | 0.491 | 0.597 | 0.597 | 0.720 | 0.780 | 0 |
| QDA | 0.192 | 0.395 | 0.516 | 0.532 | 0.672 | 0.903 | 0 |
| NNET | 0.395 | 0.493 | 0.710 | 0.627 | 0.727 | 0.798 | 0 |

The results are very much comparable between models, with some accuracy estimates going over 80%.

Table 15 below shows the so-called confusion matrix confronting predicted versus observed outcomes across cross-validation resamples for the best performance SVM2 model above. The values are proportions for each actual-predicted combination across resamples. Errors for each class are off the diagonal (about 8.6% of control samples were wrongly classified as disease samples and about 10% of the diseased samples were wrongly classified as control samples). Afterwards, a number of performance statistics are provided, including overall mean accuracy (81.4%), a 95% confidence interval for this, and sensitivity (85.4%) and specificity (71.1%) amongst others, with the diseased class corresponding to the positive outcome of the test.

TABLE 15
Confusion Matrix and Statistics
Reference

| Prediction | Diseased | Control |
|---|---|---|
| Diseased | 0.603 | 0.086 |
| Control | 0.100 | 0.212 |
| | Accuracy: | 0.814 |
| | 95% CI: | (0.801, 0.827) |

| | |
|---|---|
| No Information Rate: | 0.702 |
| P-Value [Acc > NIR]: | <2e−16 |
| Kappa: | 0.561 |
| Mcnemar's Test P-Value: | 0.0543 |
| Sensitivity: | 0.858 |
| Specificity: | 0.711 |
| Pos Pred Value: | 0.875 |
| Neg Pred Value: | 0.679 |
| Prevalence: | 0.702 |
| Detection Rate: | 0.602 |
| Detection Prevalence: | 0.688 |
| Balanced Accuracy: | 0.784 |
| ‘Positive’ Class: | Diseased |

Feline Species

The feline samples were analysed in the same was as described for the canine samples.

The following results in Table 16 and FIG. 10 summarise the predictive performance of the models.

TABLE 16
Call:
summary.resamples (object = resampsSMOTE)
Models: CPART, GLM, LDA, Bayes GLM, KNN, NNET,
QDA, SVM1, SVM2, SVM3, RF, RPART, TreeBAG
Number of resamples: 50

| | | | | | | | |
|---|---|---|---|---|---|---|---|
| Model | Min | 1^{st} Qu | Median | Mean | 3^{rd} Qu | Max | NA's |

Accuracy

| | | | | | | | |
|---|---|---|---|---|---|---|---|
| CPART | 0.333 | 0.571 | 0.667 | 0.691 | 0.833 | 1 | 0 |
| GLM | 0.500 | 0.714 | 0.817 | 0.781 | 0.857 | 1 | 0 |
| LDA | 0.286 | 0.667 | 0.714 | 0.773 | 1.000 | 1 | 0 |
| BayesGLM | 0.167 | 0.667 | 0.757 | 0.764 | 1.000 | 1 | 0 |
| KNN | 0.000 | 0.667 | 0.757 | 0.751 | 0.857 | 1 | 0 |
| NNET | 0.429 | 0.667 | 0.833 | 0.800 | 1.000 | 1 | 0 |
| QDA | 0.667 | 0.714 | 0.833 | 0.839 | 0.964 | 1 | 0 |
| SVM1 | 0.333 | 0.714 | 0.833 | 0.800 | 0.857 | 1 | 0 |
| SVM2 | 0.333 | 0.679 | 0.833 | 0.797 | 0.857 | 1 | 0 |
| SVM3 | 0.429 | 0.667 | 0.833 | 0.800 | 0.964 | 1 | 0 |
| RF | 0.429 | 0.679 | 0.833 | 0.793 | 1.000 | 1 | 0 |
| RPART | 0.286 | 0.571 | 0.667 | 0.696 | 0.833 | 1 | 0 |
| TreeBAG | 0.286 | 0.714 | 0.857 | 0.823 | 1.000 | 1 | 0 |

Kappa

| | | | | | | | |
|---|---|---|---|---|---|---|---|
| CPART | −0.400 | 0.000 | 0.276 | 0.269 | 0.565 | 1 | 0 |
| GLM | −0.286 | 0.2565 | 0.503 | 0.465 | 0.696 | 1 | 0 |
| LDA | −0.286 | 0.2589 | 0.462 | 0.494 | 1.000 | 1 | 0 |
| BayesGLM | −0.667 | 0.1989 | 0.462 | 0.445 | 1.000 | 1 | 0 |
| KNN | −0.800 | 0.0217 | 0.462 | 0.383 | 0.588 | 1 | 0 |
| NNET | −0.400 | 0.0217 | 0.571 | 0.497 | 1.000 | 1 | 0 |
| QDA | 0.000 | 0.0000 | 0.571 | 0.477 | 0.924 | 1 | 0 |
| SVM1 | 0.500 | 0.2783 | 0.571 | 0.507 | 0.696 | 1 | 0 |
| SVM2 | −0.500 | 0.2565 | 0.571 | 0.478 | 0.696 | 1 | 0 |
| SVM3 | −0.400 | 0.2500 | 0.571 | 0.494 | 0.924 | 1 | 0 |
| RF | −0.400 | 0.3000 | 0.571 | 0.526 | 1.000 | 1 | 0 |
| RPART | −0.522 | 0.0217 | 0.288 | 0.293 | 0.571 | 1 | 0 |
| TreeBAG | −0.522 | 0.3250 | 0.627 | 0.585 | 1.000 | 1 | 0 |

From the above data, it can be seen that there are not large differences between models. The best accuracies are around 80% and the best Kappa metrics are close to 60%.

Table 17 below shows the confusion matrix for the top model (TreeBAG).

TABLE 17
Confusion Matrix and Statistics
Reference

| Prediction | Diseased | Control |
|---|---|---|
| Diseased | 0.6000 | 0.0594 |
| Control | 0.1187 | 0.2219 |
| | Accuracy: | 0.822 |
| | 95% CI: | (0.775, 0.862) |

| | |
|---|---|
| No Information Rate: | 0.719 |
| P-Value [Acc > NIR]: | 1.24e−05 |
| Kappa: | 0.586 |
| Mcnemar's Test P-Value: | 0.0171 |
| Sensitivity: | 0.835 |
| Specificity: | 0.789 |
| Pos Pred Value: | 0.910 |
| Neg Pred Value: | 0.651 |
| Prevalence: | 0.719 |
| Detection Rate: | 0.600 |
| Detection Prevalence: | 0.659 |
| Balanced Accuracy: | 0.812 |
| ‘Positive’ Class: | Diseased |

The overall mean accuracy was 82.2% with a 95% confidence interval of [77.5, 86.2]%. The test sensitivity was 83.5% and the test specificity was 78.9%. Percentual errors for each class were off the diagonal. The highest was 11.9%, referring to diseased samples being identified as control samples.

From the results of Examples 1 and 2, it can be seen that the predictive models based on miRNA data are able to differentiate between control and diseased samples with around 80% accuracy for both canine and feline samples. Test sensitivity and specificity were also similar.

From the results of the above experiments, a combination of models were used to analyse the data from the FirePlex® experiments. As discussed, a number of the models gave similar results and so a combination of models produced a higher degree of accuracy in determining the presence or absence of disease.

There is therefore provided an miRNA assay to accurately identify the presence or absence of cardiovascular or heart disease in a subject (such as dogs and cats) using a biofluid such as a blood sample.

We claim:

1. A method for detecting the presence of heart disease in a subject, comprising the steps of:
(a) determining the level of expression of each of a plurality of miRNAs within a sample from a subject; and
(b) using one or more Artificial Intelligence (AI) model to predict the disease condition of the subject.

2. The method according to claim 1, wherein the one or more AI model compares the level of expression of each miRNA molecule with at least one pre-determined reference level characteristic of a non-diseased subject for each one of the plurality of the miRNA molecules of step (a), wherein a deviation of the level of expression of said miRNA molecules from step (a) in comparison with the at least one reference level allows for the diagnosis or prognosis of the disease.

3. The method according to claim 1, wherein the plurality of miRNA molecules comprises cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p or hsa-miR-486-5p.

4. The method according to claim 1, wherein the subject is an animal.

5. The method according to claim 4, wherein the subject is a cat or a dog.

6. The method according to claim 1, wherein the method further comprises the step of using a machine learning algorithm for predictive modelling.

7. The method according to claim 1, wherein the method comprises the use of a combination of AI models.

8. The method according to claim 1, wherein the method further comprises the use of at least one normaliser or control miRNA molecule.

9. The method according to claim 8, wherein the control miRNA molecule is an off-species control miRNA molecule.

10. The method according to claim 8, wherein the at least one normaliser is selected from the group consisting of hsa-miR-17-5p, cfa-miR-130b, cfa-miR-20a, cfa-miR-23a and cfa-miR-26a.

11. The method according to claim 9, wherein the at least one off-species control is selected from the group consisting of oan-miR-7417-5p, cel-mir-70-3p and ath-mir167d.

12. The method according to claim 1, wherein the disease is selected from the group consisting of dilated cardiomyopathy and related conditions, valvular disease and related conditions, endocarditis, hypertrophic cardiomyopathy and related conditions, stenosis, atrial fibrillation and other rhythm disorders, cardiac tamponade, pericardial effusion, congenital disease, or congestive heart failure, breed predispositions, parasitism, secondary conditions of other diseases, A/V node problems, toxic insults, dilation and hypertrophy.

13. The method according to claim 1, wherein the sample is a biofluid selected from the group consisting of blood, urine, milk, tissue fluid, saliva, milk, cerebrospinal fluid (CSF) or another biofluid.

14. The method according to claim 1, wherein the miRNAs are cell free miRNAs.

15. A kit for use in performing the method of claim 1 comprising means for determining the level of expression of each one of the following miRNA molecules: cfa-miR-30b, cfa-miR-30d, cfa-miR-128, cfa-miR-133a, cfa-miR-133b, cfa-miR-142, cfa-miR-206, cfa-miR-320, cfa-miR-423a, cfa-miR-499, cfa-let-7b, cfa-let-7e, hsa-let-7i-5p, hsa-miR-29a-3p and hsa-miR-486-5p.

16. A method of selecting a panel for use in disease diagnosis comprising the steps of:
(a) selecting a group of miRNA molecules the differential expression of which may be associated with a disease condition;
(b) training one or more AI model to be able to predict the disease condition; and
(c) using the one or more AI model to reduce the number of miRNAs in the panel to a minimum number to provide a panel of miRNAs that still produces a result.
